(19) United States
(12) Patent Application Publication
(10) Pub. No.: US 2017/0100574 A1
(43) Pub. Date: Apr. 13, 2017

(54) Apparatus configured for accurate up-loading of a single dose onto a delivery applicator

(71) Applicant: Edward Tak Wei, Berkeley, CA (US)

(72) Inventor: Edward Tak Wei, Berkeley, CA (US)

(21) Appl. No.: 14/756,769

(22) Filed: Oct. 9, 2015

(51) Int. Cl. A61M 35/00; A61F 9/00; A61K 31/66

(52) U.S. Cl. CPC A61M 35/003; A61K 31/66; A61F 9/0017

(57) ABSTRACT

The body has a number of sensitive surfaces that are subject to injury by physical, chemical and biological agents. The results of injury are, for example, signs of inflammation such as redness, swelling, feeling of heat, pain and loss of function, and discomfort such as itching, irritation, burning sensations, and dysesthesia. The nasal membranes, the ocular surface, the lips and gingiva, and the genital surfaces are examples of such sensitive surfaces. Topical agents can be delivered to these surfaces using pre-medicated single unit applicators, such as swabs, wipes, or cotton-tips attached to handles, but for high frequency use, there is an advantage to using a reservoir bottle of medicated solution together with a packet of single applicators. In this invention, an apparatus embodiment for topically delivering a pharmaceutical comprises a specially designed container, together with a package of single applicators. The desired parameters of drug delivery, namely, precise dosage, hygienic use and storage of the drug solution, single use, and economy for high frequency of use, are provided by the container design. The container accurately up-loads a fixed volume of solution onto the tip of a single applicator; wherein the tip of the applicator is made of an absorbent material such as cotton or rayon. The aperture or opening of the container has a cap that is air-tight when sealed. The diameter of the container aperture is not wider by more than 20% of the maximum width of the applicator tip. The container has a reservoir as an internal capacity to hold 5 to 50 mL of a liquid, and more preferably 5 to 15 mL. At the bottom of the container's reservoir there is a cylindrical cavity, the center of which is aligned perpendicularly to the center of the aperture. The lower wall of the container's reservoir is shaped to funnel the liquid contents of the container to the bottom of the cylindrical cavity formed as a reservoir pocket. This pocket has a concave upward, saucer like, or hemispheric shape to accommodate the tip of the applicator. The length of the container's cylindrical cavity is sufficient to sheath at least 50% of the body of applicator. This apparatus can up-load a single unit applicator with a low coefficient of variation in the up-loaded volume.

### BACKGROUND OF THE INVENTION

Field of the Invention

The present invention pertains generally to the field of a method of preparing a pharmaceutical solution for topical delivery and an apparatus therefore. More specifically the present invention pertains to the configuration of an apparatus or container that will permit accurate loading of solutions onto singly used applicators, such as cotton-tipped applicators or swabs. Such applicators are then used for topical delivery of solutions onto bodily areas such as the nasal membranes, the ocular surface, the lips and gingiva, the genital surfaces, and the skin.

Description of Related Art

The body's surfaces are exposed to the external environment and these surfaces are subject to injury by physical, chemical and biological agents. The results of tissue injury are the cardinal signs of inflammation (redness, swelling, feeling of heat, pain and loss of function) accompanied by symptoms of discomfort, such as itching, irritation, burning sensations, and dysesthesia. The surface of the skin has a layer of dead cells, called the stratum corneum, composed of keratin proteins that are tough and water-impermeable and protects the underlying tissues. For example, the heel has on average 86 cell layers in the stratum corneum. By contrast, the penile shaft, nasolabial folds (smile lines), and eyelids have 6, 7, 8 cell layers, respectively. The nasal membranes and the ocular surfaces have sparse keratin and must be kept moist by glandular (serous and mucous) secretions in order to avoid desiccation. For the skin, tinctures, gels, pastes, ointments, creams, lotions, oils, foams, sprays, mists, or aerosols may be used for topical drug delivery. But for the nasal cavity surfaces, the ocular surface, the lips and gingiva, and the genital skin, a more accurate and localized drug delivery method is required. Ideally, for these sensitive surfaces, the delivery vehicle is an aqueous solution and the method must permit:
precise dosage, for example, the delivered volume must be within ±5%
hygienic use and storage of the drug solution, so it is not easily contaminated
the applicator to be used for delivery is preferably disposable and singly used
the method of delivery should be economically sustainable for high frequency of use, for example, for use of 3 or more applicators per day.

Several pre-medicated swabs that meet the first three criteria above are currently available. These are single-use sealed units wrapped in materials which are relatively impermeable to liquids and do not dry out (for example, a polystyrene case). Examples of these pre-medicated swab applicators in individual units are known as SwabDose™ from Unicep Corporation (1702 Industrial Drive, Sandpoint, Id., USA) and Pro-Swabs™ from American Empire Manufacturing (3828 Hawthorne Court, Waukegan, Ill., USA). Each tip of the applicator (e.g., 40 to 100 mg of cotton) is saturated by being encased with 0.5 to 1.5 mL of a solution. The unit is packaged in an elegant individual container. Although pricing information cannot be obtained precisely, the range is $0.10 to $0.25 per unit, with each box containing from 12 to 25 units. These costs are acceptable for delivery of prescription drugs, but less attractive for over-the-counter consumer products that require high frequency of usage.

A pre-medicated swab for topical application of solutions can be manufactured without a rigid container. For example, a sophisticated device is made by the S & B. Co., Ltd. Masan-Si, Korea (see www.snbglobal.com). The apparatus, called a “Magic Bar” maintains a solution by capillary action in a capped polyethylene tube above a cotton tip. Twisting off the cap of tube containing the solution allows the liquid to descend by gravity from the reservoir onto the cotton tip and the solution is now on the tip for delivery. The swab tip is not enclosed. But single units can be wrapped in moisture-proof cellophane with a peelable seal and thus kept clean and hygienic. A similar system is made by Unidose Systems, Inc. located in Rancho Cucamonga, Calif. 91730 and described at www.unidose.com. Proposed uses of the Unidose applicators are pre-medicated swabs for makeup removal (eye and mascara), lipstick removal, vitalizing the lips, cuticle conditioners, nail polish remover, and swabs for applying compounds such as antifungal drugs, or antiseptics such as iodine.

The delivery units using solutions stored by capillary action in hollow tubes, in single cellophane wrappers, are less expensive to make than units singly encased in polystyrene. One limitation of the “capillary system” applicators is the volume of solution that can be stored by capillary retention, without inadvertent leakage of the solution onto the absorbent tip. Currently, the Unidose system has a fill volume of 0.15 mL per unit. This may not be adequate when the delivered volume for a given application has to be larger. Although pricing information cannot be obtained precisely, the range for the units based on capillary action is probably $0.03 to $0.10 per unit, with each box containing from 12 to 25 units.

### BRIEF SUMMARY OF THE INVENTION

In this invention, an apparatus embodiment for topically delivering a pharmaceutical comprises a specially designed container, together with a package of single applicators. The desired parameters of drug delivery, namely, precise dosage, hygienic use and storage of the drug solution, single use, and economy for high frequency of use, are provided by the container design. The container accurately up-loads a fixed volume of solution onto the tip of a single applicator; wherein the tip of the applicator is made of an absorbent material such as cotton or rayon. The aperture, or mouth, of the container has a cap that is air-tight when sealed. The diameter of the container aperture is not wider by more than 20%, and preferably not more than 10%, of the maximum width of the applicator tip. The container has a reservoir as an internal capacity to hold 5 to 50 mL of a liquid, and more preferably 5 to 15 mL. At the bottom of the container's reservoir there is a cylindrical cavity, the center of which is aligned perpendicularly to the center of the aperture. The lower wall of the container's reservoir is shaped to funnel the liquid contents of the container to the bottom of the cylindrical cavity formed as a reservoir pocket. This pocket has a concave upward, saucer like, or hemispheric shape to accommodate the tip of the applicator. The length of the container's cylindrical cavity is sufficient to sheath at least 50% of the body of applicator. This apparatus can up-load a single unit applicator with a low coefficient of variation in the up-loaded volume.

### BRIEF DESCRIPTION OF THE SEVERAL VIEWS OF THE DRAWINGS

FIG. 1. is an illustration of an apparatus designed with a flip-top cap and an airtight seal, and the center of the aperture is aligned perpendicularly with a cavity at the bottom of a container.

FIG. 2a. is an illustration of a container embodiment filled with liquid and an applicator that is used with the unit.

FIG. 2b. is an illustration of the applicator inserted into the sheath of the bottle's bottom cavity and the loading of the applicator tip with liquid.

FIG. 3. is a graph on the use of an applicator and a cooling agent for the relief of rhinorrhea. The abscissa shows the number of tissues deposited into a basket by an individual each day for 9 days. C3, 2 mg/mL, was applied twice a day onto nasal membranes and the number of tissues was then recorded daily for another 9 days. The data are shown as the mean±SD.

FIG. 4. is a graph showing the improvement in computer vision syndrome symptoms scores after daily 4× use of an applicator to the eyelids of a solution containing C3, 2 mg/mL, for two weeks. The symptoms, ranked on a scale of 0 to 6, were fatigue, burning, dryness, blurred vision, and dullness of vision. Data of the averaged score are for n=20 subjects per group, *P<0.05, and **P<0.01.

FIG. 5. is a graph showing the comparison of loading volumes of applicators inserted into three types of bottles. Boston R=Boston Rounds, 7.5 mL bottles; Scint V=Scintillation vials, 20 mL bottles; DRB=designed reservoir bottle (container) of this invention, 5.8 mL. The coefficient of variation is equal to the standard deviation divided by the mean expressed as percent, and is a measure of dispersion of a frequency distribution.

### DETAILED DESCRIPTION OF INVENTION

An apparatus embodiment of the invention is adapted for topically delivering a pharmaceutical as at least one biologically active dose, more preferably to deliver a plurality of single doses over a period of time.

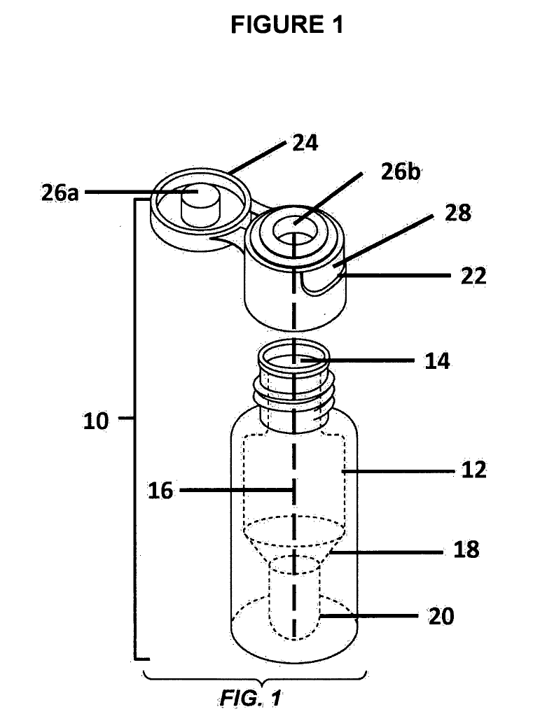

Turning to FIG. 1, part of an apparatus embodiment is illustrated wherein a container 10 is adapted to hold liquids. Container 10 can conveniently be made of glass or plastic. The container 10 defines therein a reservoir 12 of sufficient size to hold at least one dose of a biologically active pharmaceutical in liquid form. The container 10 has a sealable opening, aperture or mouth, 14 and defines a longitudinally extending axis 16. The reservoir 12 and the opening 14 are aligned along the axis 16. The reservoir 12 and the opening 14 each have a dimension outward from the axis but with the reservoir having the greater width. The reservoir 12 includes a funnel portion 18 and a pocket 20 which each are disposed a spaced distance along the axis from the opening 14 and each are extending symmetrically outward from the axis 16, wherein the funnel 18 provides liquid communication between the reservoir 12 and the reservoir pocket 20 when at least one dose of pharmaceutical is within the reservoir;

A cap 22 is adapted to seal the container opening 14 in an air-tight configuration. The cap 22 is designed to permit access into the container for multiple applicators which are loaded with the desired quantity of pharmaceutical while avoiding contamination. As illustrated by FIG. 1, a particularly preferred cap embodiment has a flip top lid 24 with a male member 26a that snugly fits into and seals ringed donut hole 26b. A groove 28 can be used to facilitate opening the lid 24 with a thumb.

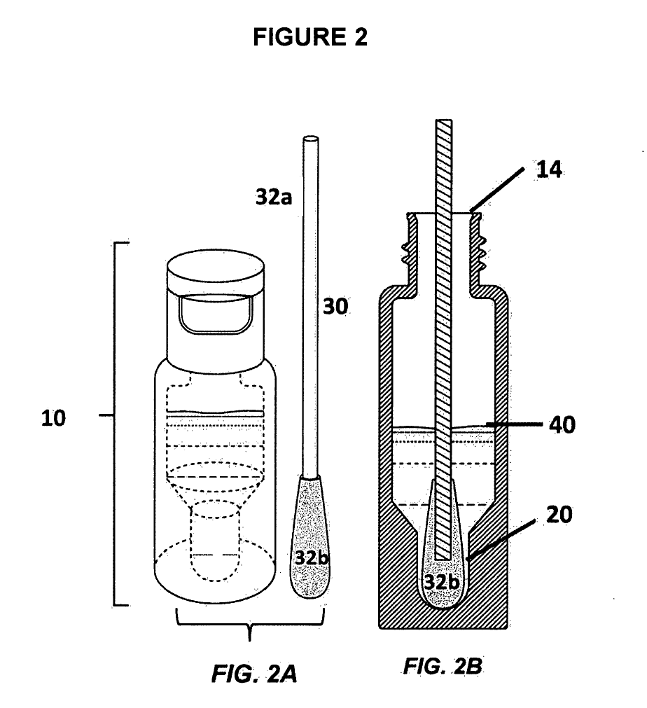

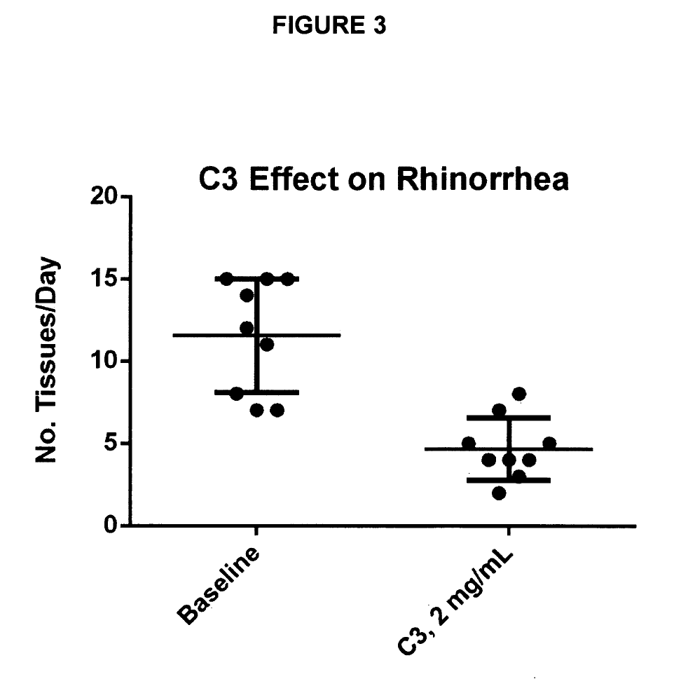

Turning to FIG. 2A, the apparatus embodiment further comprises at least one applicator 30. Applicator 30 is formed by a rod 32 longitudinally extending between ends 32 a, b with a quantity of absorbent material being carried at rod end 32b. The longitudinal extension of the rod 32 is greater than the longitudinally extending axis 16 of the container 10. Turning to FIG. 2B, the rod end 32b with absorbent material is adapted to fit relatively snugly within the reservoir pocket 20 when the applicator 30 is inserted through the container opening 14, along the container axis 16, past the funnel 18 and into the reservoir pocket 20. As illustrated by FIGS. 2A and 2B, there will be present within reservoir 20 at least one biologically active dose 40 of a pharmaceutical in liquid form.

FIGS. 2A and 2B illustrate an apparatus embodiment with one applicator 30; however, in a particularly preferred embodiment, the delivery unit will comprise the container 10 together with a package of single applicators. The container 10 stores the liquid formulation of the active ingredient (e.g. 5 to 15 mL per bottle). The individual disposable applicators each will have ends or tips 32b of suitable absorbent materials such as cotton, polyamide (10% Nylon)-polyester, or rayon (70%)-polyester (30%). For example, Puritan 803-PCL applicators are cotton-tipped applicators attached to a 3-inch (˜7.5 cm) polystyrene rod that are ideal for delivery of solutions onto surfaces such as the nasal membranes, ocular margins, and the gingiva and lips. Bigger containers and tips are readily available for larger surfaces such as genital skin. The applicator is singly inserted into the reservoir bottle, saturated with drug solution, applied to target, and then disposed with instructions not to re-insert the applicator into the container in order to avoid contamination. The bottle may be made of glass or plastic such as polyethylene or polypropylene.

### A. Designed Bottle (Container)

Thus, in summary, components of an inventive embodiment are shown in the FIG. 1, FIGS. 2a, and 2b:

Bottle. A portable container for holding liquids, characteristically having a neck and mouth, and made of glass or plastic.

Aperture. The opening of the bottle is fitted with an air-tight cap. The center of the opening is perpendicularly aligned with the center of the bottom of the reservoir pocket. The width of the aperture is slightly wider, e.g. by 3 to 20%, than the widest part of the applicator.

Reservoir. This part of the bottle holds the liquid to be delivered and has a capacity of 5 to 50 mL.

Funnel. The bottom part of the reservoir is shaped as a funnel to direct the liquid towards the bottom of the reservoir pocket.

Reservoir pocket (cylindrical cavity). The pocket is a cylindrical cavity at the bottom of the container that is designed to form a sheath around at least 50% of the tip of the applicator and has a concave upward bottom to accommodate the tip. A preferred shape of the reservoir pocket is cylindrical. The reservoir pocket forms a snug fit for the applicator tip such that insertion results in optimal absorption of the liquid from the reservoir.

Applicator. An applicator preferably is a “swab” in form of a rod (preferably 3 inches) made of plastic (e.g. polystyrene) or wood to which is attached an absorbent material (preferably cotton or rayon) of a predetermined mass (e.g. 40 to 100 mg of cotton). The absorbent material carries the active ingredient which is dissolved in a liquid. When loading the tip of the applicator, the handle is inserted vertically to the bottom of the reservoir pocket. The handle of the applicator may be marked with a colored ring or bar to show the maximum depth of insertion of the handle into the bottle. When the delivery apparatus comprises a package of single applicators, they may be wrapped together in one bunch or separately from each other.

Liquid. A substance capable of flowing freely like water, and which is not a solid or gas.

Aqueous solution. The active ingredient is dissolved in water and the liquid is free-flowing.

The tip of the applicator is inserted to the bottom of the reservoir bottle. After loading, the rod serves as a handle to manipulate the absorbent material to a correct position for application. The dimensions and mass of the absorbent material on the tip of the applicator is important for proper delivery. The absorbent material is saturated because it is bathed in the liquid solution: so the amount of fluid up-loaded and off-loaded is determined by the mass of the absorbent material. Too little up-load, and not enough solution is delivered; too much up-load, and the solution coalesces to form drops which may run off the delivery target. For the nasal membranes and ocular surface, it was found by experiment, that a cotton or rayon mass of 40 to 100 mg per tip was optimal for a delivered volume of 8 to 40 μL to the target tissues. For the gingiva, oral cavity, or genital skin, a larger mass on the tip may be desirable: for example, a mass of 60 to 250 mg per tip and a delivered volume of 30 to 100 μL to the target tissues.

Currently a bottle with a single applicator for repeated use is available for applying mascara, nail polish, and for antiseptics such as iodide tinctures. In such items, contamination of the parent solution with repeated insertions into the bottle and exact dosage per administration are not stringent requirements for correct usage. Examples of suppliers for components of a delivery system, e.g. glass bottles or snap-on screw-on hinge closures, are Primaral Glass (India) and Zeller Plastik (Global Closure Systems, Germany), respectively.

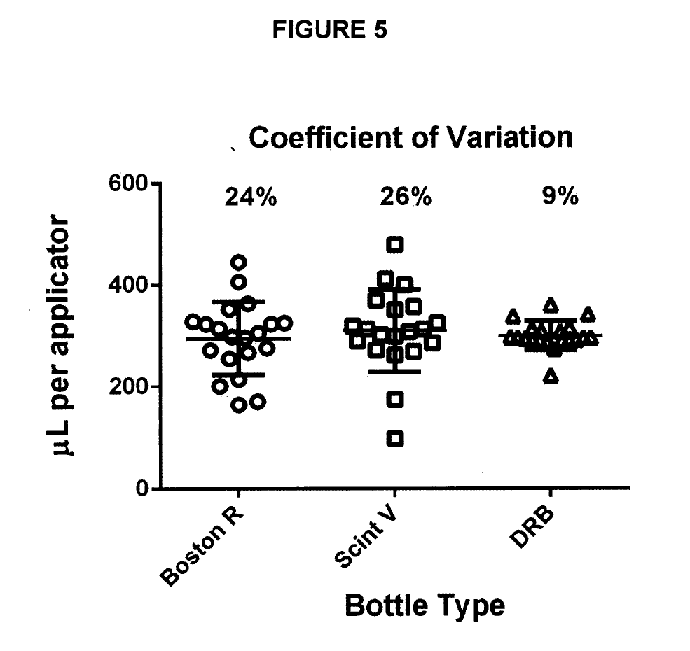

In the present invention, it is recognized that an applicator should only be inserted once to be fully loaded. A key design feature is the reservoir pocket. If the bottom of the container is flat or rounded, the up-loaded volume of solution into the applicator becomes variable when the solution levels drop. This fact is shown in the Example (FIG. 5). The advantage of the current reservoir pocket design is that full saturation of the applicator tip is consistently obtained until the solution is nearly all used up. This feature of reproducible dosing is important for solutions that have medical applications because regulatory requirements are such that the dose has to be specified, accurate, and within a certain range. The aperture width of the cap is also important. An aperture much wider than the applicator tip is undesirable because large droplets may be picked up and retained by the applicator tip when it is withdrawn from the bottle. The excess volume may fall off the tip before it reaches target or flood the target. A wide aperture adds variability to dosing.

A designed bottle, or container 10, holding multiple doses, together with a plurality of applicators 30, can be viewed as an applicator kit, which has lower costs per unit because once a mold (hollow matrix) has been created for the prototype then plastic bottles can be produced at low costs. Of the three systems for single disposable applicators: namely, swabs encased in polystyrene, single units based on capillary retention of the solution in a tube, and a designed bottle with applicators, the bottle is economical. For applications with a large number of users, e.g. swabs for relief of nasal irritants from air pollutants, or for computer vision syndrome or dry eye, or, the costs of the product are a significant factor for consumer acceptance.

### B. Applications of the Designed Bottle

For the purposes of description, the item shown in the FIG. 1, 2a, 2b will sometimes herein be described as a “Designed Reservoir Bottle” or abbreviated as DRB, together with the applicator.

### Nasal Irritation

The nose serves as a conduit for 10,000 to 12,000 L of air per day into and out of the respiratory tract. When the nasal membranes are irritated, it is readily perceived as an uncomfortable condition. Inflammation of the nasal mucosa caused by air pollutants, allergens, or infections such as the common cold virus, will generate a sense of “stuffiness”, increased secretions, resistance to inspiration, or a feeling of nasal obstruction. Irritation from air pollutants is a common complaint in congested and polluted cities. Popular remedies in China and SouthEast Asia are inhaled vapors of camphor, menthol, and eucalyptus oil (e.g. Tiger Balm and Poy Sian Inhaler). The pharmacological relief of nasal symptoms is a mass market. Another example of nasal dysfunction is infection by the common cold virus. For example, the frequency of the common cold in the US is estimated to be about one cold per person per year. The estimated cost to the US economy is $40 billion a year. Yet another category of nasal dysfunction, allergic rhinitis, affects 20 to 40 million people each year in the US. Nasal congestion diminishes the quality of life: for example, the patient cannot sleep, is tired and anxious, and is hindered from normal social activities.

I have found that a class of water soluble compounds have cooling properties and will relieve nasal discomfort when applied with a cotton-tipped applicator in the nasal vestibule. This technology is described in U.S. Ser. No. 14/545,014, published Sep. 24, 2015, and hereby incorporated by reference. Cryosim-3 (C3) is an example of a prototype molecule that acts by soothing and cooling the surface of the nasal membranes. Delivery of C3 is achieved with a cotton-tipped applicator saturated with a C3 solution, 0.2 to 0.3% in distilled water. The applicator is put just inside the nasal opening and the solution dabbed on. Relief from the sense of congestion is immediate (<5 min) and long-lasting (5 hr+).

Cryosim-3 acts as a TRPM8 agonist. TRPM8 is a physiological receptor protein present on nerve endings associated with detection of sensations caused by heat abstraction (cooling). Standard TRPM8 agonists are I-menthol and icilin but C3 differs from these compounds (and others) because it is water-soluble and easy to deliver, has a long duration of action, and does not irritate the sensitive nasal surfaces. The target sites are located on the nasal epithelium, on the nerve endings at the base of the epithelial layer. A DRB mechanism of delivery will allow C3 to be used by the general population at low costs.

For application to the nasal cavity, the individual is instructed to gently insert the tip of the applicator into the opening of the nostrils (the nasal vestibule), and, after removal of the tip, to gently compress the nostrils with the thumb and forefinger towards the center of the nose. The instructions for application may include teaching the individual to repeat application, or “topping up”, to ensure that sufficient composition is delivered to the target. Once the subject has learned what to expect, the individual can adjust the dosage (e.g., by dabbing more liquid at the medial site), as needed, to achieve the desired effect. It has been observed that individuals learn how to effectively apply the cooling agent after one or two trials and can do so without difficulty.

### Computer Vision Syndrome

The computer vision syndrome (CVS) is a condition of eye fatigue, tiredness, dryness, blurring, and headache caused by too much (>3 hr) continuous use of computer, tablet, e-reader, or cell phone. Related terms for CVS are: Digital Eye Strain and Asthenopia, and this condition is recognized and defined for treatment by the American Optometric Association. The causes of CVS are multifactorial: for example, not blinking and the eye surface becomes dry; fatigue of the eye muscles for focus and movement; poor lighting and posture when using the eyes; and poor vision. When the brain cells are hyperactive, this can also cause fatigue and tension. Approximately 80+% of US and 50+% of China use the Internet. Relief of CVS is a mass market item that should be available to everyone.

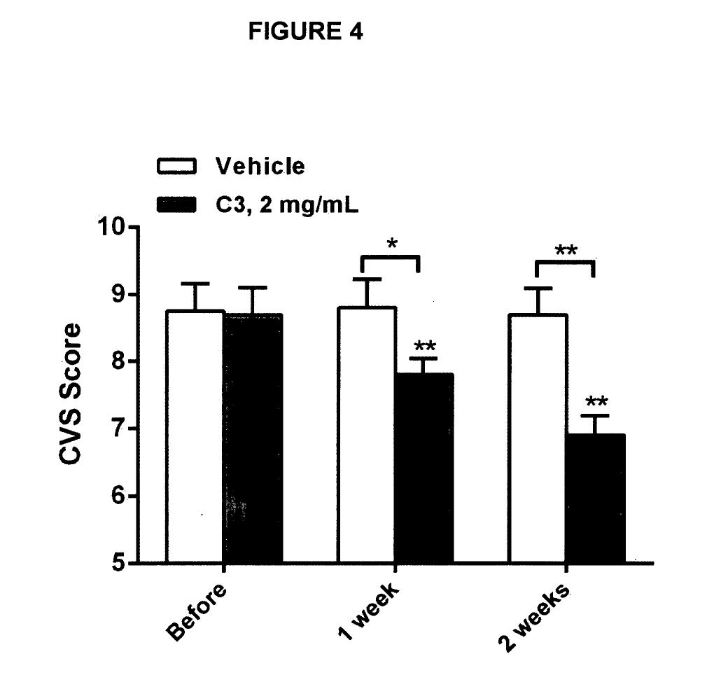

As shown in the Example (FIG. 4), a study has shown that solutions of cooling agents, for example, 1-dialkylphosphoryalkanes (TRPM8 agonists), when applied to the upper eyelids with a cotton-tipped applicator, produce a refreshing cooling sensation on the ocular surface, facilitate a clearer vision, make the eyes comfortable and relieves CVS. A particularly effect agent is called C3 (U.S. Ser. No. 13/999,979, Oct. 14, 2015). The DRB is a delivery system that can be used for the treatment of CVS and related ocular conditions of discomfort.

### Oral Cavity and Gingiva

Two surfaces in the oral cavity, the gum (gingiva) and mouth lining, e.g. inner lip and buccal mucosa, are subject to inflammation and pain. Gingivitis and periodontitis are common conditions of gum discomfort. Another condition, aphthous stomatitis (canker sores) is a condition of small ulcerations on the non-keratinizing surfaces of the mouth such as the inner lip and buccal mucosa. A cooling solution, or an antibiotic such as doxycycline (for the gingiva), can be directed to these surfaces with an applicator to relieve discomfort or infections. The DRB method will facilitate frequent and inexpensive use.

### Genital Skin

Lichen sclerosus is an inflammatory condition primarily of genital skin. It is a rare disorder associated with intense itching (pruritus), pain, and dysesthesia (burning sensations). Traditional dermatological vehicles such as creams and lotions cannot be applied to genital skin because non-aqueous solvents irritate. An aqueous solution applied with a neutral absorbent material, such as cotton or rayon is ideal for delivery. The absence of the use of excipients minimizes the likelihood of further tissue irritation. The DRB is ideal for controlling drug delivery because the bottle size and tip size can be easily modified to the target tissues: for example, by making the reservoir bottle larger (e.g. from 10 to 30 mL) and making the delivery tip wider (e.g. from 0.25 inch to 0.5 inch or 1 inch). Such adaptations can be done at low costs and the DRB optimized for treating a distressing disease such as lichen sclerosus.

### C. Examples of Drug Action

### Nasal Discomfort

A 50-year old distinguished physiologist with MD PhD degrees had perennial rhinitis, with daily rhinorrhea, of unknown cause. He could estimate the degree of rhinorrhea by the number of Kleenix tissues he deposited into the waste basket each day, on average about 10 per day. At first, he was sceptical about the potential of C3, but tried it for a week at 2 mg/mL and “thought” that it worked. He thereby decided to do a “real” trial, by stopping use of C3 and returning to “baseline”. For nine days, he averaged 12 tissues per day. Then he started using C3 (FIG. 4)) twice a day, and noticed that the number of Kleenix tissues dropped to 5 tissues per day (P<0.01) for the subsequent 9 days. He was using a Q-Tip with a small tip, so he was asked to try 3 mg/mL instead of 2 mg/mL. He said that the subjective relief of nasal congestion was better with 3 mg/mL. He continues use of C3 and to record the number of tissues used each day. When introduced to the concept of a designed bottle with a package of applicators, he was most enthusiastic and said that this will greatly add to the convenience of using C3 in controlling nasal congestion and secretions.

### Computer Vision Syndrome and Ocular Discomfort

This study was conducted by Dr. J. M. Yang and Professor K. C. Yoon, both ophthalmologists at Chonnam University, Gwangyu, Korea. A total of 40 subjects with mild dry eye symptoms agreed to participate. Unicep Swabdose™ units, containing 1 mL of C3 at 2 mg/mL in purified water or water vehicle only was given to the subjects (n=20 per group) and the recommended usage was for four times a day. The study was for 2 weeks, with a single applicator unit to be used per day. The average volume per application off-loaded, each time the applicator was used, averaged 20 μL to both eyes, or 20 μg of C3 per eye for a 2 mg/mL solution. A questionnaire to assess the symptoms of computer vision syndrome was administered at the first week and at the second week of use. Subjects were asked to rank fatigue, burning, dryness, blurred vision, and dullness of vision on a scale of 0 to 6 (0=“no symptom”, 6=“very severe symptom”). The average aggregate score for the two test groups are shown in the FIG. 4. The CVS-type of symptoms was significantly improved at both 1 and 2 weeks after C3. No significant adverse effect such as ocular pain, irritation, or discomfort was reported from both groups during the study.

It was noted that although the design of the experiment required that a single applicator be used for four times a day, this was inconvenient because the solution of the Swabdose™, once opened, can be spilled or lost if the unit is not maintained in a vertical position. Also, frequency of re-use could not be controlled, so a single applicator may be used for more than one day and this prolonged use will increase the risk of contamination of the applicator or the container. A DRB will remove these limitations of the single Swabdose unit for drug delivery.

### OTHER APPLICATIONS

In principle, any ocular drug used as eye drops can be adapted to the DRB and swabs because of convenience of use. The wiping method of application is more “patient-friendly” than eye drops. Some applications, however, stand out as examples. Demulcents, or polymer “lubricants” such as carboxymethylcellulose, hypromellose, polyethylene glycol 400, hyaluronan, and propanediol(s) increase the elastoviscous properties of the ocular fluids (usually this can be achieved with ophthalmic solutions in the range of 25 to 50 centipoises) and are widely used for the sensation of “dry eye”. A DRB containing such lubricants will have utility.

The selected active pharmaceutical ingredient (API) should be evenly dispersible in a liquid composition so that during manufacture an uniform solution can be produced under clean or sterile conditions. For purposes of formulation, the API should preferably be miscible or soluble in aqueous solutions at neutral pH and/or isotonicity and not adhere as a particle to the absorbent substrate. The aqueous solubility of an API will facilitate meeting requirements of sterility, a unit dose dispenser, uniform dose delivery, and formulations free of preservatives. Examples of ocular drugs that may be incorporated into swabs include: cyclosporine, antihistamines such as olopatadine, α-adrenergic agonist vasoconstrictors such as phenylephrine, naphazoline, or tetrahydrozoline, anti-inflammatory drugs such as diclofenac, anti-viral agents, antibiotics, or cooling agents such as menthol or WS-12.

### D. Experimental Study of DRB Reliability

A sketch of a prototype DRB was prepared by MDG Worldwide Ltd. (KwunTong, Hong Kong, SAR), a company specializing in graphics design. The following dimensions were selected for the prototype: a 55 mm tall container with a 20 mm diameter circular base, and a 16 mm diameter flip-top cap. The upper side of the cap had a 5 mm long 6.5 mm diameter male cylindrical plug which fitted a corresponding “donut” hole of 6.5 mm diameter on the lower side of the cap. The donut hole was elevated by 2 mm above the surface of the cap. A finger groove on the cap facilitated opening of the flip-top cap. The body of the bottle or reservoir was 38 mm tall (17 mm for the lid and cap). At 15 mm from the bottom there was a funnel-shaped receptacle fitted with a reservoir pocket, as show in the FIG. 1, 2a. For this particular prototype, the funnel was 5 mm high, and the reservoir pocket was 12 mm deep, with the bottom of the cylindrical cavity a concave upward hemisphere with a 8 mm diameter. The drawing was given to HK3DPrint (Kwai Chung, Hong Kong, SAR), a professional 3D printing service, who then prepared a 3D CAD file (computer assisted design file) and printed a PET (polyethylene) plastic prototype. This prototype was used in the following experiment.

To compare the relative efficiency of the DRB versus other containers for up-loading solutions, 3.2 mL of water was placed in a Boston Rounds ¼ oz. (7.5 mL) natural LDPE Bottles (E.D. Luce Packaging #N1315), or a scintillation vials, 20 mL with 20 mm cap size (Wheaton, #986704) or the DRB. A Puritan 803-PCL applicator with a 3-inch polystyrene attached to an extra absorbent cotton tip was inserted to the bottom of the containers, removed, and the up-loaded amount of water was determined by the difference in the weight of the container. This was repeated 10 times per container for two containers, and the results shown in FIG. 5. The mean±SD up-loaded volume per applicator was 295±72 μL, 310±81 μL, and 300±28 μL for the Boston Rounds, scintillation vial, and DB, respectively. The coefficient of variation (ratio of the standard deviation to the mean), a standardized measure of the dispersion of a frequency distribution, was 24%, 26%, and 9% for the three containers, respectively. Thus, the DRB container provides a more reliable up-loading mechanism.

The broader base of the Boston Round more often gave low values when the fill volume dropped below 35%. The wider aperture of the scintillation vial sometimes gave higher values because large droplets adhered to the applicator when it was removed from the container. Both of these events, under-loading and over-loading are undesirable because there may be insufficient drug to achieve an effect (e.g. nasal cavity cooling, relief of ocular discomfort) or the excess volume may cause irritation (e.g. the droplets will coalesce on the eyelids and access the corneal surface and cause pain). The DRB significantly reduces variation in dosing, and this is attributed to the configuration of the container.

1. An apparatus adapted for topically delivering a pharmaceutical as at least one biologically active dose, comprising:
a container, the container defining therein a reservoir of sufficient size to hold at least one dose of a biologically active pharmaceutical in liquid form, the container having a sealable opening and defining a longitudinally extending axis, the reservoir and the opening being aligned along the axis, the reservoir and the opening each having a dimension outward from the axis, the reservoir defining a funnel and a pocket each disposed a spaced distance along the axis from the opening and each extending symmetrically outward from the axis, wherein the funnel provides liquid communication between the reservoir and the reservoir pocket when at least one dose of pharmaceutical is within the reservoir;
a cap adapted to seal the container opening in an air-tight configuration;
at least one applicator, the at least one applicator formed by a rod longitudinally extending between ends with a quantity of absorbent material being carried at one rod end, the longitudinal extension of the rod being greater than the longitudinally extending axis of the container, the rod end with absorbent material being adapted to fit relatively snugly within the reservoir pocket when the applicator is inserted through the container opening, along the container axis, past the funnel and into the reservoir pocket; and,
at least one biologically active dose of a pharmaceutical in liquid form.

2. The apparatus as in claim 1 wherein the at least one dose is disposed in the container with the cap assembled so as to be releasably sealing the container.

3. The apparatus as in claim 1 wherein the at least one applicator is a plurality of applicators each being capable of assembly with the container by insertion along the container axis, wherein the absorbent material of the applicator is symmetrically disposed around the rod end and defines an applicator tip, the pharmaceutical is within the container reservoir in an amount equivalent for delivering an active dose via at least most of the applicators, and wherein the cap is releasably sealing the container with the pharmaceutical therein.

4. The apparatus as in claim 3 wherein the container opening is not greater than about 20% of the maximum width of the applicator tip.

5. The apparatus as in claim 3 wherein the reservoir has a capacity of holding from about 5 mL to about 50 mL of liquid pharmaceutical.

6. The apparatus as in claim 3 wherein the longitudinally extending axis of the applicator is not greater than about 50% of the longitudinally extending axis of the axis defined from the container opening to a portion of the reservoir pocket farthest from the container opening.

7. The apparatus as in claim 6 wherein the reservoir pocket has a conical shape to which the applicator tip conforms when inserted to the furthest depth of the container.

8. The apparatus as in claim 3 wherein the pharmaceutical contains a topical ocular drug or a topical nasal drug.

9. The apparatus as in claim 3 wherein the pharmaceutical contains one or more of a cooling agent, a 1-dialkylphosphorylalkane, or a demulcent polymer lubricant.

10. The apparatus as in claim 3 wherein the plurality of applicators and container having from about 5 mL to about 50 mL of liquid pharmaceutical therein comprise a delivery kit with at least most of the applicators capable of delivering a consistent dose of liquid pharmaceutical when each is inserted into the container with the rod end fit snugly within the reservoir pocket.
